(19) United States
(12) Patent Application Publication
(10) Pub. No.: US 2022/0111163 A1
(43) Pub. Date: Apr. 14, 2022

(54) RESPIRATORY VENTILATION DEVICE

(71) Applicant: SLEEPINNOV TECHNOLOGY, Moirans (FR)

(72) Inventors: Nikolaï Hungr, Moirans (FR); Jérôme Argod, Moirans (FR)

(73) Assignee: SLEEPINNOV TECHNOLOGY, Moirans (FR)

(21) Appl. No.: 17/428,161

(22) PCT Filed: Jan. 31, 2020
(86) PCT No.: PCT/EP2020/052457

§ 371 Date: Nov. 3, 2021

(30) Foreign Application Priority Data
Feb. 4, 2019 (FR) FR1901084

(51) Int. Cl. A61M 16/00

(52) U.S. Cl. CPC A61M 16/0069; A61M 16/0875

(57) ABSTRACT

A device for assisting respiration (e.g., a respirator) is configured to send an air flow, generated by a fan, into a conduit. The conduit extends between the respirator and a respiratory mask, configured to be used by a user. The respirator comprises an enclosure extending around a longitudinal axis. The enclosure comprises an air inlet configured to admit air into the enclosure. An upstream air circulation zone extends between the air inlet and the fan. An air outlet is configured to be connected to the conduit so that, when the fan operates, air flows in series from the air inlet through the upstream air circulation zone. The fan is arranged in a ventilation chamber. The ventilation chamber is maintained by a first flexible membrane and by a second flexible membrane extending at a non-zero distance from the first membrane.

### CROSS-REFERENCE TO RELATED APPLICATIONS

This application is a national phase entry under 35 U.S.C. § 371 of International Patent Application PCT/EP2020/052457, filed Jan. 31, 2020, designating the United States of America and published as International Patent Publication WO 2020/161019 A1 on Aug. 13, 2020, which claims the benefit under Article 8 of the Patent Cooperation Treaty to French Patent Application Serial No. FR1901084, filed Feb. 4, 2019.

### TECHNICAL FIELD

The technical field of the disclosure is a continuous positive pressure ventilation device. This type of device is commonly used in the treatment of sleep apnea.

### BACKGROUND

The use of continuous positive pressure (CPP) ventilation is a reference treatment in the field of sleep apnea. This treatment involves continuously blowing air into a mask applied on the face of the user. It can involve a nasal, nostril or facial mask. The blown air reaches the respiratory channels of the user, by exerting sufficient pressure thereon, so as to prevent a collapse from forming.

The positive CPP ventilation devices are used at night. Consequently, they must be as quiet as possible, on the understanding that there are two noise sources:
the propagation of mechanical vibrations generated by the fan: this involves vibrations that are likely to propagate through the solid parts of the device, in particular the walls; and
the flow of air through the device: upstream of the fan the air is sucked in, whereas downstream of the fan the air is blown.

Thus, the noise is either of mechanical or of aeraulic origin.

Moreover, the device can be intended to be transported and it is important for it to be compact enough to be placed in luggage. It also must be easy to use. Furthermore, the manufacturing cost must be reduced: therefore, the design of the device must be as simple as possible.

Among the devices known in the art, the device described in EP 2923721 can be cited, for example. This device is intended to be arranged on a horizontal support. However, the horizontal footprint of this device is relatively large, in particular when the device comprises a humidification module. A more compact device has been described in EP 2822626. It comprises a ventilation chamber that is retained inside an enclosure by an elastomer membrane. The elastomer membrane extends between a periphery, fixed to the enclosure of the device, and the ventilation chamber. The ventilation chamber is inserted into an opening provided at the center of the membrane. In the central part, the membrane is divided into a lower part and an upper part, so as to form a casing extending on either side of the ventilation chamber. At the periphery of the membrane, the membrane comprises a bellows so as to attenuate a transmission of vibrations, generated by the ventilation chamber, toward the rigid enclosure. In order to prevent contact between the ventilation chamber and the enclosure, cones are provided that allow any impacts during axial movements of the ventilation chamber to be absorbed. The division of the membrane, the presence of a bellows and the arrangement of cones form elements that make manufacturing the membrane more complex, and that accentuate the fragility of the membrane. Document U.S. Pat. No. 7,975,688 also describes a ventilation chamber retained by an elastomer membrane.

Among the devices known in the art, document U.S. Pat. No. 8,453,640, which describes a compact respiration device, also can be cited.

### BRIEF SUMMARY

A ventilation respiration device has been designed by particularly focusing on noise reduction during operation, whether this involves mechanical vibrations caused by the operation of the fan or noise from the air flow. Furthermore, the device that is the subject matter of the disclosure is compact, robust and easy to manufacture. Its design makes it easy to transport.

An aim of the disclosure is a respiratory ventilation device intended to send an air flow, generated by a fan, into a conduit, with the conduit extending between the device and a respiratory mask intended to be used by a user, the device comprising an enclosure extending around a longitudinal axis and comprising:
an air inlet intended to admit air into the enclosure;
an upstream air circulation zone extending between the air inlet and the fan;
an air outlet, the air outlet being configured to be connected to the conduit, so that, when the fan operates, air successively flows from the air inlet through the upstream air circulation zone, the fan and the air outlet, the device being such that:
the fan is arranged in a ventilation chamber; and
the ventilation chamber is retained by a first flexible membrane extending between a first periphery and the ventilation chamber, the first periphery being fixedly retained relative to the enclosure, the first membrane defining a first central opening, through which the ventilation chamber, or a support connected to the ventilation chamber, extends.

The device can comprise one of the following features, taken individually or according to the possible technical combinations:
the ventilation chamber is retained by a second flexible membrane, separate from the first membrane, the second membrane extending between a second periphery and the ventilation chamber, the second periphery being fixedly retained relative to the enclosure at a non-zero distance from the first periphery, the second membrane comprising a second central opening, through which the ventilation chamber, or a support connected to the ventilation chamber, extends;
the ventilation chamber comprises an intake portion, a fan housing, a discharge portion and a motor compartment;
the first membrane extends between the first periphery and the fan housing or between the first periphery and the motor compartment;
the second membrane extends between the second periphery and the intake portion;
the device comprises a third membrane extending between a third periphery, which is fixed relative to the enclosure and/or is fixed relative to the shell, and the discharge portion; and
the device comprises a shell, which preferably is rigid, extending around a central axis, the shell being arranged in the enclosure by advantageously being fixed relative thereto, the shell extending around the ventilation chamber, the shell comprising an intake opening allowing air to be admitted into the shell, and an extraction opening allowing air to be extracted from the shell.

Thus, the upstream air circulation zone extends:
outside the shell, between the air inlet of the enclosure and the intake opening;
inside the shell, between the intake opening and the ventilation chamber;
the shell comprises a shell bottom opposite the intake opening. The shell bottom can face the intake portion of the ventilation chamber, with the shell bottom forming an air deflector between the intake opening and the intake portion;
the central axis of the shell corresponds to the longitudinal axis; in this case, the intake opening and the shell bottom are preferably aligned along the longitudinal axis;
the first membrane is retained by the shell and extends between the shell and the ventilation chamber, with the first membrane at least partly extending perpendicular to the central axis of the shell;
the first membrane forms a first partition inside the shell, with the first membrane comprising an opening so as to allow air to pass on either side of the partition;
the second membrane is retained by the shell and extends between the shell and the ventilation chamber, with the second membrane at least partly extending perpendicular to the central axis of the shell;
the second membrane forms a second partition inside the shell, with the second membrane comprising an opening so as to allow air to move on either side of the partition;
the discharge portion of the ventilation chamber:
   emerges in the extraction opening of the shell; or
   extends through the extraction opening of the shell;
the device comprises an upstream expansion chamber, adjacent to the intake opening of the shell, the upstream expansion chamber comprising an inlet, a central part and an outlet arranged around an upstream axis, with the section of the central part, perpendicular to the upstream axis, being greater than the respective sections of the inlet and of the outlet of the upstream expansion chamber. The upstream expansion chamber can emerge at the intake opening of the shell;
the device comprises a downstream expansion chamber, adjacent to the intake portion of the ventilation chamber, the downstream expansion chamber comprising an inlet, a central part and an outlet arranged around a downstream axis, with the section of the central part, perpendicular to the downstream axis, being greater than the respective sections of the inlet and of the outlet of the downstream expansion chamber;
the device is such that the downstream expansion chamber emerges at the intake portion of the ventilation chamber;
the device is such that the second membrane extends between the second periphery and the downstream expansion chamber, so that the intake portion is retained by the second membrane, and
the first membrane and the second membrane are flexible and are formed from an elastomer material. The same is the case for the potential third membrane.

Further advantages and features will become more clearly apparent from the following description of particular embodiments of the disclosure, which are provided by way of non-limiting examples, and are shown in the following figures.

### BRIEF DESCRIPTION OF THE DRAWINGS

FIG. 1A is a diagram showing a use of the device.

FIGS. 1B and 1C are views of the enclosure of the device.

FIG. 1D shows part of the enclosure of the device, from which a detachable flap has been removed.

FIG. 1E shows components of the device located inside the enclosure.

FIGS. 2A and 2B show a shell located in the enclosure, as well as an intake tube and an outlet tube for air.

FIG. 2C shows an example of an upstream expansion chamber.

FIG. 2D shows the ventilation chamber.

FIG. 2E shows the first membrane, the second membrane, as well as the third membrane, with these membranes allowing the ventilation chamber to be retained inside the shell.

FIG. 2F shows the ventilation chamber, as well as a downstream expansion chamber.

FIG. 2G shows the first membrane, the second membrane, as well as the third membrane, with these membranes allowing the ventilation chamber to be retained inside the shell.

FIG. 3A shows the first membrane.

FIG. 3B shows the second membrane.

FIG. 4A shows the main components of the enclosure, through which air flows. The same is the case for FIG. 4B.

FIG. 5 is another embodiment of the device.

### DETAILED DESCRIPTION

Throughout the following description, the terms upstream/downstream are to be understood according to the direction of the air flow.

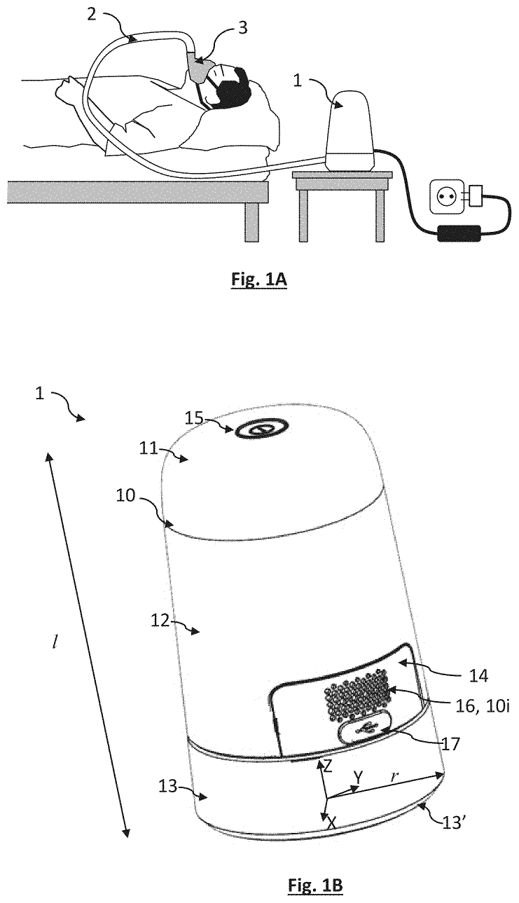

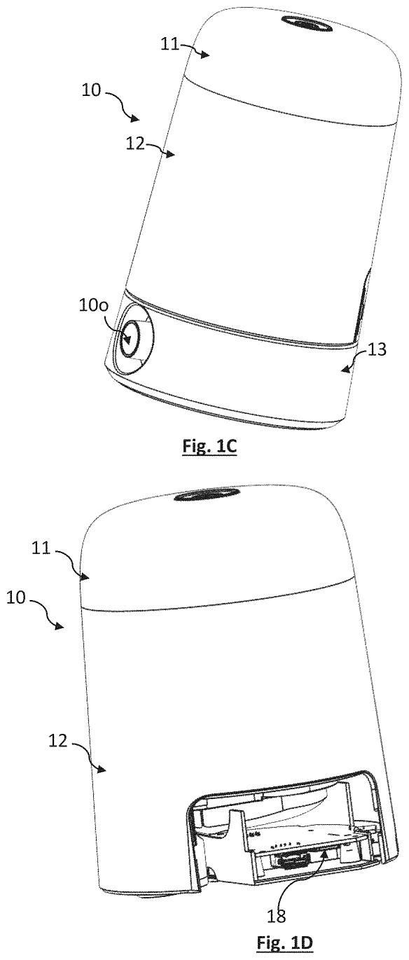

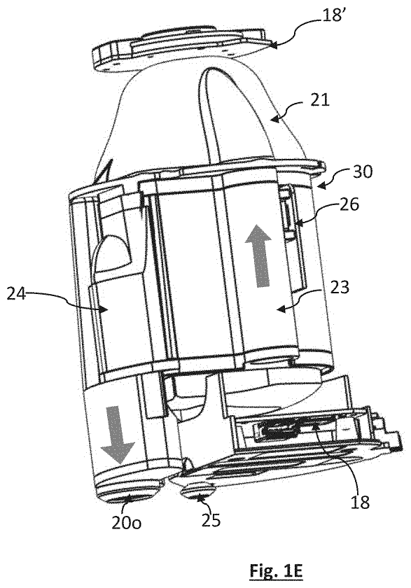

FIG. 1A shows a device 1 for assisting respiration according to embodiments of the disclosure. The device comprises a conduit 2 connecting it to a respiratory mask 3 intended to be applied on the face of a user. Preferably, the conduit 2 is a flexible conduit, which is a few meters long. The device 1 comprises a fan, controlled to maintain a setpoint pressure at the respiratory mask. It is basically intended for nocturnal use. During its use, the device particularly can be placed on a flat support, for example, a bedside table. To this end, its horizontal footprint must be minimized.

FIGS. 1B and 1C show an enclosure 10 of the device 1. The enclosure 10 extends along a longitudinal axis Z by defining a length l. It extends radially along a base plane XY, in which it defines a radius r. The length l typically ranges between 10 cm and 40 cm, whereas the radius r generally ranges between 5 cm and 30 cm. The dimensions of the device make it easy to transport. Moreover, the device is adapted to be placed on a small flat support, for example, a table. During use, the longitudinal axis Z is preferably parallel to the vertical, whereas the base plane XY is horizontal.

The enclosure 10 is preferably made up of a rigid material, for example, a plastic. In the example shown, the enclosure 10 comprises an upper section 11, an intermediate section 12 and a lower section 13. The lower section is demarcated by a lower surface 13′, which is preferably flat and parallel to the base plane XY. The intermediate section 12 comprises a grill 16, acting as a filter, and forming an air inlet 10i, through which air can enter inside the enclosure 10. The intermediate section 12 also comprises a connection socket 17 allowing the device 1 to be connected to a remote processing unit, so as to configure a control unit 18 arranged inside the enclosure 10 and described with reference to FIG. 1D.

In this example, the connection socket 17 and the air inlet 10i are arranged on a detachable flap 14.

An on/off control switch 15 is arranged on the upper section 11. The arrangement at the top of the enclosure 10 makes it easy to access by a user, including in half-light or darkness. The control switch 15 can comprise a light source, for example, a light emitting diode, so that it can be seen in the dark. The enclosure 10 is preferably symmetrical around the longitudinal axis Z. The on/off control switch is then centered around the longitudinal axis Z. Thus, irrespective of the rotation of the device 1 around the longitudinal axis Z, the on/off switch 15 does not move: its position is independent of the rotation of the device Z around the longitudinal axis Z.

FIG. 1C shows that the lower section 13 comprises an outlet opening 10o forming an air outlet of the device 1. The outlet opening 10o is intended to be connected to the conduit 2, shown in FIG. 1A, when the device 1 is used.

According to a variant, the lower section 13 comprises a water container, forming a humidifier. The air blown by the device 1 flows, in the lower section 13, above the water container so as to humidify upstream of the opening 10o. The arrangement of the humidifier inside the lower section avoids modifying the spatial requirement in the plane XY.

FIG. 1D shows the intermediate section 12 and the upper section 11. In FIG. 1C, the flap 14 has been removed. A control unit 18 can be seen that allows the device 1 to be configured and/or controlled, and, in particular, allows the fan to be controlled. The control unit 18 can be configured by a wired connection connected to the socket 17, as previously described.

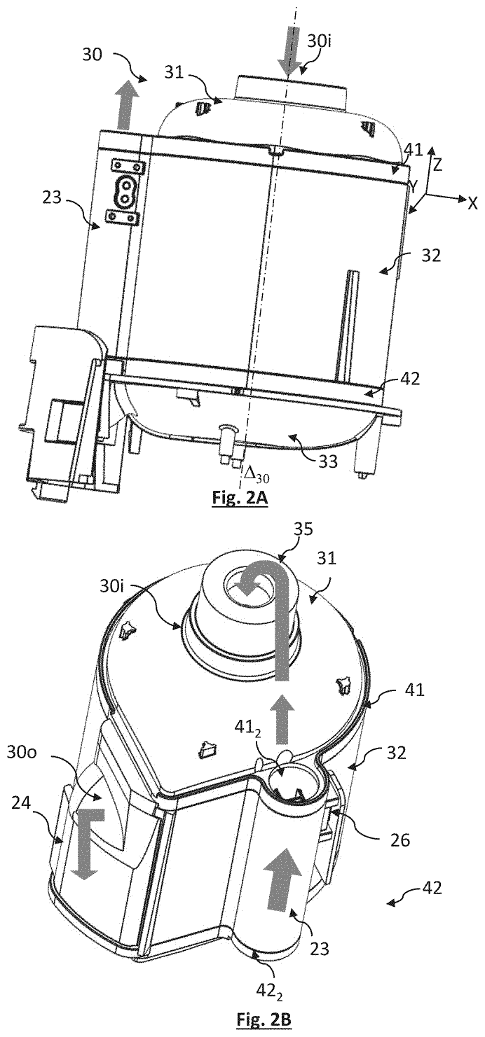

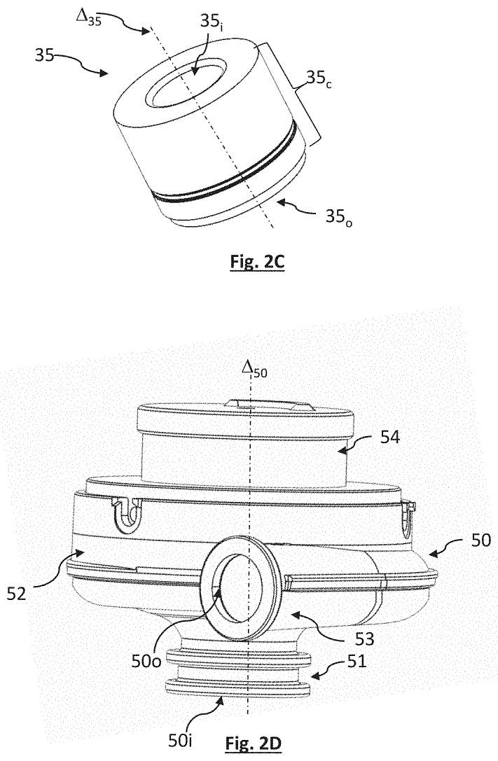

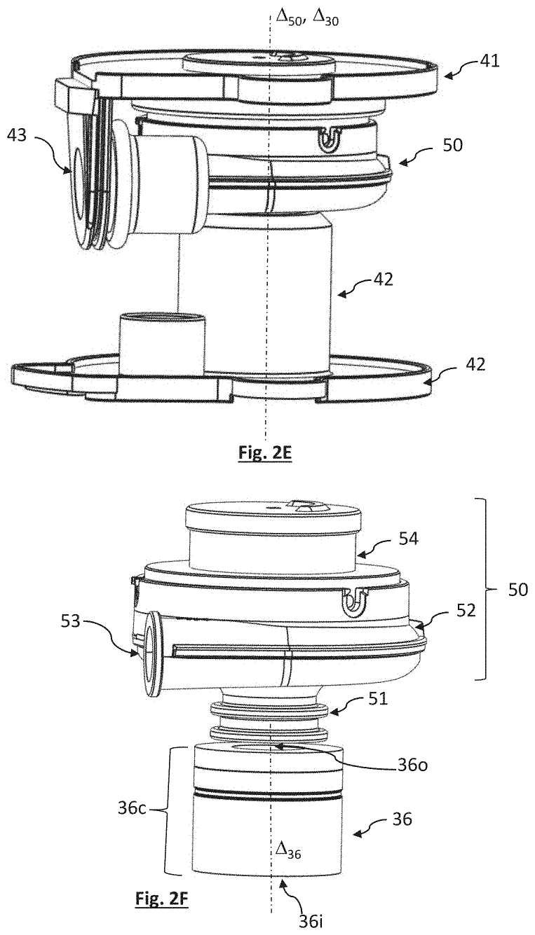

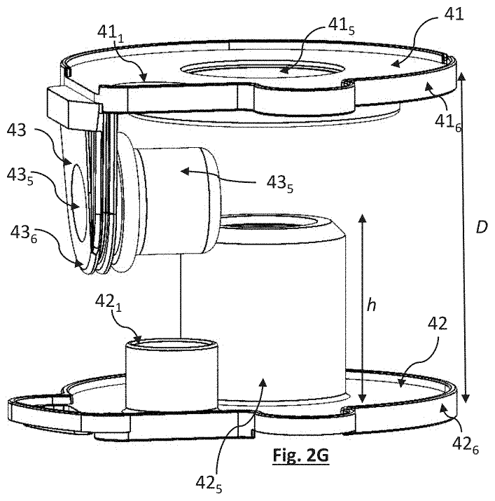

FIG. 1E shows the inside of the intermediate section 12 and of the upper section 11. The air admitted into the enclosure 10, through the opening 10i, flows through an inlet tube 23, toward the top of the device, parallel or substantially parallel to the longitudinal axis Z. Substantially parallel is understood to mean parallel, whilst allowing for an angular tolerance of ±20° or of ±10°. The inlet tube 23 extends along a shell 30, described with reference to FIGS. 2A to 2F. The inlet tube 23 emerges in a space demarcated by the shell 30 and an upper cover 21. The shell 30 and the upper cover 21 are made from a preferably rigid material, for example, a plastic. FIG. 1E also shows an outlet tube 24 extending along the shell 30, through which air flows toward the air outlet 10o provided in the lower section 13. The outlet tube 24 emerges, in the lower section 13, via an opening 20o. The opening 20o is provided at the interface between the intermediate section 12 and the lower section 13. The air, emerging in the lower section 13 via the opening 20o, flows, in the lower section 13, toward the outlet opening 10o. The device also comprises an auxiliary electronic board 18′, connected to the start-up switch 15. The auxiliary electronic board 18′ is also connected to the control unit 18. The device 1 comprises a flow meter 26, connected to the control unit 18. The flow meter 26 allows the air flow in the inlet tube 23 to be measured. In this example, the flow meter 26 is provided at the inlet tube 23. The flow meter 26 allows adjustment of a pressure setpoint of the air that is directed by the device 1 toward the mask 3 of the user. For example, in the case of apnea, the air flow drops, which causes the setpoint pressure to increase.

The device comprises a pressure sensor 25 measuring the air pressure in the lower section 13, upstream of the outlet opening 10o. Depending on the measured pressure, the control unit 18 adapts the power of the fan so as to maintain a pressure that is as stable as possible around the setpoint pressure.

An important element of the disclosure is the presence of a shell 30, arranged inside the enclosure 10. Thus, the enclosure 10 completely surrounds the shell 30. The shell 30 extends around a central axis Δ₃₀. Preferably, the central axis Δ₃₀ is parallel, even coincident, with the longitudinal axis Z along which the enclosure 10 extends. FIGS. 2A and 2B are external views of the shell 30. In the example shown, the shell 30 extends, along the central axis Δ₃₀, between a first opening 30i, allowing air to be admitted inside the shell, and a solid shell bottom 33. The shell comprises an upper part 31, an intermediate part 32, as well as the shell bottom 33, assembled pairwise, along the central axis Δ₃₀.

A first membrane 41 extends between the upper part 31 and the intermediate part 32, forming a seal between the upper part 31 and the intermediate part 32. The first membrane is a flexible membrane, produced, for example, from an elastomer material, for example, a silicon-based material. A second membrane 42 extends between the intermediate part 32 and the shell bottom 33, forming a seal between the intermediate part 32 and the shell bottom 33. The second membrane 42 is a flexible membrane, produced, for example, from an elastomer material, for example, a silicon-based material.

The inlet tube 23 and the outlet tube 24 form a single part with the intermediate part 32. This allows the number of parts forming the device to be reduced.

The air emerging from the inlet tube 23 flows into a space demarcated by the upper part 31 and the upper cover 21. It enters the shell 30 via the intake opening 30i, as shown by the grey arrows in FIGS. 2A and 2B. The air is extracted from the shell 30 via a second opening, forming an extraction opening 30o, provided in the intermediate part 32 of the shell 30. The extraction opening 30o is shown in FIG. 2B. It extends parallel to the central axis Δ₃₀.

Advantageously, an expansion chamber, called upstream expansion chamber 35, extends at the intake opening 30i. The term expansion chamber denotes a chamber extending between a chamber inlet and a chamber outlet, along an axis. As shown in FIG. 2C, the expansion chamber 35 is such that it comprises a central part 35c between the inlet chamber 35i and the outlet chamber 35o, so that, perpendicular to the axis Δ₃₅ of the chamber, denoted using the term upstream axis, the section of the central part is strictly greater than the inlet section and the outlet section of the chamber. A section is understood to be the surface demarcated by the expansion chamber, perpendicular to the axis around which the chamber extends. Thus, the section of the central part is at least 1.5 times bigger, even at least two or three times bigger than the section of the inlet and of the outlet of the expansion chamber. The central section can be partially filled with an acoustic foam, for example, an open pore foam. The use of such an expansion chamber allows the air flow noise to be significantly reduced.

More generally, the use of air circulation through sections comprising wide portions followed by narrow portions allows the air flow noise to be reduced.

FIGS. 2D and 2E show the main elements arranged inside the shell 30. The shell 30 contains a ventilation chamber 50, in which a fan extends. The fan is the essential accessory of the device, since it ensures the circulation of air between the inlet 10i of the enclosure and the outlet 10o. More specifically, the fan allows air to be sucked in through the inlet 10i of the device and allows air to be blown from the outlet 10o of the device. The ventilation chamber 50 comprises four parts in this example:
a tubular shaped intake portion 51 allowing air to be admitted into the ventilation chamber through an inlet 50i;
a round housing 52 surrounding the fan, with the blades thereof rotating inside the round housing;
a tubular shaped discharge portion 53 emerging at an outlet 50o of the ventilation chamber 50; and
a motor compartment 54 electrically connected to the control unit 18.

The housing 52 and the discharge portion 53 extend perpendicular to the intake portion 51. The housing 52, the intake portion 51 and the motor compartment 54 extend around an axis Δ₅₀, which corresponds to the axis of rotation of the fan.

Preferably, the axis of rotation Δ₅₀ of the fan is coincident with the longitudinal axis Z of the enclosure 10 and/or with the central axis Δ₃₀ of the shell 30. Thus, the axis of rotation Δ₅₀ of the fan corresponds to the longitudinal axis Z of the enclosure. Consequently, when the longitudinal axis Z of the enclosure 10 is vertical, or substantially vertical, which corresponds to the operating position of the device 1. The blades of the fan extend in a horizontal plane and rotate along the plane, inside the housing 52.

A noteworthy aspect of the device is that it thus comprises three parts nested in one another, namely:
the enclosure 10, which extends around the longitudinal axis Z;
the shell 30, included in the enclosure 10 and extending around the central axis Δ₃₀, and which is included in the enclosure 10; and
the ventilation chamber 50 comprising the fan, which extends around the axis of rotation Δ₅₀ of the fan, and which is included in the shell.

Such “Russian doll” type nesting allows a series of baffles to be formed in order to force the air to follow a series of zigzags between the inlet 10I of the enclosure 10 and the fan. Such air circulation, inside the enclosure 10, allows a particularly quiet device to be obtained. It also should be noted that the respective inlets of the enclosure 10, of the shell 30 and of the ventilation chamber 50 are not aligned. Thus, the inlet 10i of the enclosure, which forms the inlet of the device 1, is arranged on a lateral face of the enclosure, parallel or substantially parallel to the longitudinal axis Z. The intake opening 30i of the shell 30 is provided around the longitudinal axis Z, in a plane perpendicular or substantially perpendicular to the central axis Δ₃₀, with the axis preferably being coincident with the longitudinal axis Z. The upper cover 21 is located opposite the inlet (intake opening 30i) of the shell 30, which cover is solid. The upper cover 21 forms an air deflector. The air flow emerging from the inlet tube 23 is therefore forced into a first rotation of 180° in order to engage in the shell 30. The air flow is then drawn in toward the ventilation chamber 50, the inlet 50i of which is oriented around the axis of rotation Δ₅₀, with the axis preferably being aligned with the central axis Δ₃₀ and with the longitudinal axis Z. The inlet 50i of the ventilation chamber 50 is arranged opposite the shell bottom 33, which being solid forms an air deflector. Thus, the air flow is forced into a second 180° rotation so as to engage through the inlet 50i of the ventilation chamber.

Nesting the shell 30 inside the enclosure 10 also allows the noise generated by the device to be attenuated, with the enclosure 10 attenuating the noise generated inside the shell 30.

In order to limit the noise and to attenuate the mechanical vibrations caused by the operation of the fan as much as possible, the ventilation chamber 50 is kept suspended by the first membrane 41 and the second membrane 42. The first membrane 41 and the second membrane 42 are formed from a flexible material, typically an elastomer, for example, a silicon-based material. The purpose of these membranes is threefold:
to retain the ventilation chamber while attenuating the vibrations of the fan, parallel to the axis of rotation or perpendicular thereto: this improves the quiet operation of the device and allows contact between the ventilation chamber and the shell to be avoided;
to define the air circulation inside the chamber 35, upstream of the ventilation chamber, with the membranes having openings 41₁, 42₁ forming passages through which the air flow flows; and
to form seals between the various parts forming the shell 30, respectively between the upper part 31 and the intermediate part 32, and between the intermediate part 32 and the shell bottom 33.

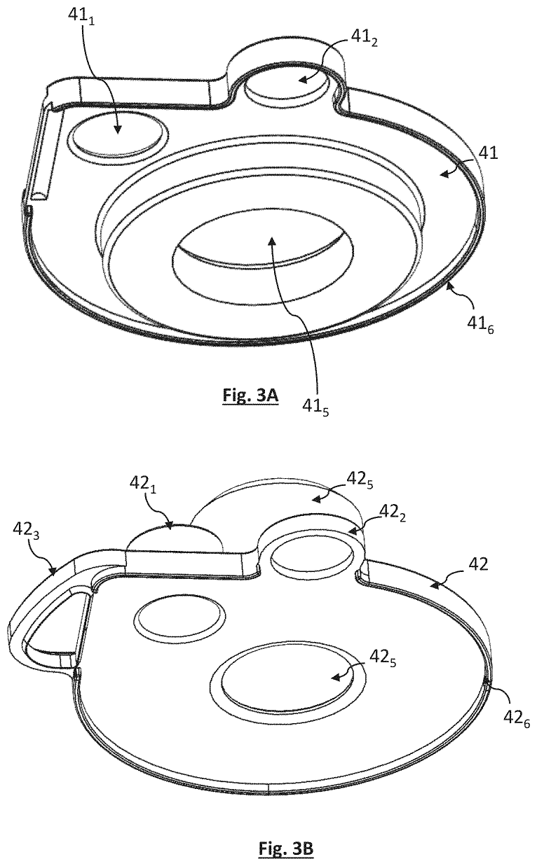

FIG. 2E shows the arrangement of the first membrane 41 and of the second membrane 42 inside the shell 30. As shown in FIGS. 2E, 2G and 3A, the first membrane 41 extends between a periphery 41₆, called first periphery, fixed to the shell 30, and the ventilation chamber 50. The ventilation chamber 50 is inserted into a first central opening 41₅. The first periphery 41₆ is retained between the upper part 31 and the intermediate part 32 of the shell 30. The first central opening 41₅ allows the ventilation chamber 50 to be inserted and retained. When the ventilation chamber 50 is inserted, the first central opening 41₅ is resiliently deformed and adjusts itself around the ventilation chamber 50, and, more specifically, around the housing 52 or the motor compartment 54. In this embodiment, the first central opening 41₁ is in direct contact with the ventilation chamber 50, even though it is possible to arrange a support between the first central opening 41₅ and the ventilation chamber 50. The retention of the ventilation chamber 50 using the first membrane 41 is provided by the tension of the first membrane 41, between the first periphery 41₆ and the first central opening 41₅. Thus, the first membrane 41 acts as a support for the ventilation chamber. The first membrane 41 extends perpendicular to the axis of rotation Δ₅₀, as well as to the central axis Δ₀ and to the longitudinal axis Z, between the ventilation chamber 50 and the first periphery 41₆.

As shown in FIGS. 2E, 2G and 3B, the second membrane 42 extends between a periphery 42₆, called second periphery, connected to the shell 30, and the ventilation chamber 50, and, more specifically, the intake portion 51 of the ventilation chamber 50. The second periphery 42₆ is retained between the intermediate part 32 and the shell bottom 33. The second membrane 42 comprises a central opening 42₅, called second central opening. The second central opening 42₅ allows the support of the ventilation chamber 50 to be inserted and retained. The retention of the ventilation chamber is provided:
either directly by the second membrane 42, with the membrane extending in contact with the ventilation chamber 50, and, in particular, at the intake portion 51, which corresponds to the example shown;
or by a support, inserted between the second membrane 42 and the intake portion 51 of the ventilation chamber 50.

In this example, the device 1 comprises an expansion chamber 36, inserted through the second central opening 42₅ of the second membrane 42. The expansion chamber 36, denoted using the term downstream expansion chamber, is similar to the previously described upstream expansion chamber 35. It comprises a central part 36c, between a chamber inlet 36i and the chamber outlet 36o, such that, perpendicular to a downstream axis Δ₃₆, the section of the central part 36c is strictly greater than the inlet section and the outlet section of the chamber. The outlet 36o of the downstream expansion chamber engages in or around the intake portion 51 of the ventilation chamber 50. As for the upstream expansion chamber 35, the downstream expansion chamber 36 helps to reduce the air flow noise. In the example shown, the upstream axis Δ₃₅ and the downstream axis Δ₃₆ are parallel to the central axis Δ₃₆ of the shell 30.

The example shown in this description comprises two expansion chambers 35 and 36 arranged in series upstream of the ventilation chamber. According to other embodiments, a single expansion chamber could be used. However, it is considered that the presence of two expansion chambers enables more effective reduction of the noise due to the air flow.

The second central opening 42₅ of the second membrane 42 is tubular. It extends, along the axis of rotation Δ₅₀ (or the central axis Δ₃₀), at a height h typically ranging between 5 mm and 6 cm, for example, 3.5 cm. This enables better gripping of the expansion chamber 36. The height h is shown in FIG. 2G.

In the vicinity of their respective peripheries 41₆ and 42₆, the first membrane 41 and the second membrane 42 are spaced apart, along the central axis Δ₃₀, by a distance D preferably ranging between 1 cm and 5 cm. The distance D is shown in FIG. 2G. The peripheries 41₆ and 42₆ form points for attaching the ventilation chamber 50 on the shell 30. With the shell 30 being fixed relative to the enclosure 10, the first and second peripheries are fixed relative to the enclosure 10. Keeping a distance D between these attachment points promotes good retention of the ventilation chamber and good attenuation of the vibrations of the ventilation chamber caused during the operation of the fan. It is also noted that retaining the ventilation chamber using two flexible membranes that are spaced apart from each other, at least at their respective peripheries, avoids the use of membranes comprising one or more bellows. It is known that such bellows enable effective attenuation of the vibrations, but they form a particular area of weakness and increase the manufacturing cost.

The retention of the ventilation chamber 50 using two membranes that are spaced apart from each other, in the vicinity of their respective peripheries, allows the movement of the ventilation chamber parallel to the central axis Δ₃₀ of the shell 30 to be limited. This avoids, for example, any impact between the ventilation chamber 50 and a rigid part of the device. This also allows an impact of the fan with the rigid walls of the shell 30 to be avoided when the device is transported. This design therefore allows a robust device to be obtained. This avoids the presence of damping means arranged on the ventilation chamber, as described with reference to the prior art.

The device comprises an optional third membrane 43, extending essentially parallel to the central axis Δ₃₀ of the shell 30. It comprises a central opening 435, called third central opening, engaging around the discharge portion 53 of the ventilation chamber 50. The third membrane 43 thus forms a seal between the discharge portion 53 of the ventilation chamber 50 and the outlet tube 24. The air thus passes from the discharge portion 53, through the extraction opening 30o of the shell 30, toward the outlet tube 24. In the example shown in FIG. 2G, the third membrane 43 has a tubular portion 435 engaging around the discharge portion 53. This allows the ventilation chamber 50 to be retained, while attenuating the vibrations thereof under the effect of the operation of the fan. The third membrane 43 has a periphery 436, called third periphery 436, secured to the shell 30. More specifically, the third periphery is inserted through the extraction opening 30o of the shell 30. The third membrane 43 can be secured to the first membrane 41 or separate from the membrane.

FIGS. 3A and 3B, respectively, show the first membrane 41 and the second membrane 42. In addition to the previously described peripheries 41₆, 42₆ and the central openings 41₅, 42₅, the membranes comprise other openings acting on the air circulation inside the shell. The first membrane 41 comprises an opening 41₁ extending into the shell 30 and forming an air passage. In this part of the shell 30, the first membrane 41 forms an internal partition extending perpendicular to the central axis Δ₃₀ of the shell 30. The opening 41₁ forms an air restriction, with the air flowing on either side of the first membrane 41, which helps to attenuate the noise of the air flow. Similarly, the second membrane 42 comprises an opening 42₁ extending into the shell 30 and forming an air passage. In this part of the shell 30, the second membrane 42 forms an internal partition extending perpendicular to the central axis Δ₃₀. This opening forms an air restriction, with the air flowing on either side of the second membrane 42, which helps to attenuate the noise of the air flow.

In addition to the openings described above, the first and second membranes define other structural openings, for conforming to the structure of the device 1.

Thus:
the first membrane 41 comprises a structural opening 412 extending around the inlet tube 23 in the vicinity of the interface between the inlet tube 23 and the upper cover 21. The first membrane 41 in this case acts as a seal between the inlet tube 23 and the upper cover 21;
the second membrane comprises a first structural opening 422 extending around the inlet tube 23 in the vicinity of the interface between the intermediate part 32 of the shell 30 and the shell bottom 33; and
the second membrane comprises a second structural opening 423 extending around the outlet tube 24.

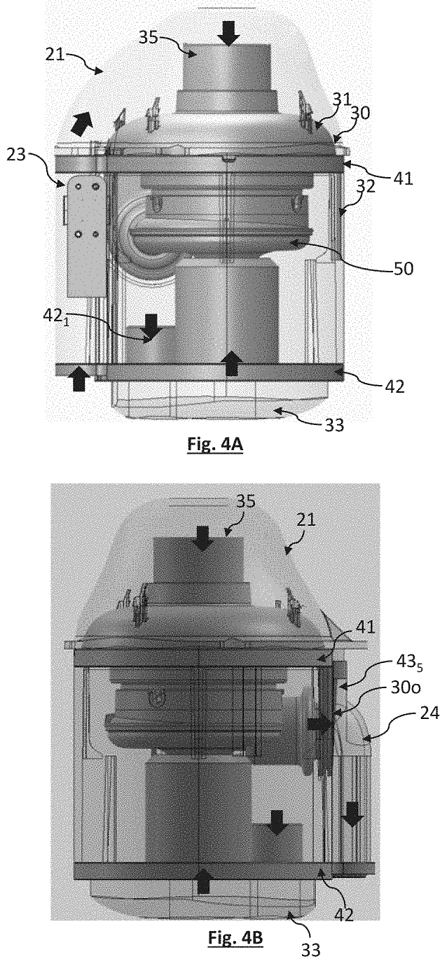

FIGS. 4A and 4B show the air circulation inside the enclosure, with the circulation being shown by black arrows: after having been admitted into the enclosure 10, the air flows through the inlet tube 23, then enters the upstream expansion chamber 35 after having been deflected by the upper cover 21. It then enters inside the shell 30 and subsequently flows through the openings 41₁ and 42₁, respectively, provided in the first and second membranes 41 and 42. It is subsequently deflected by the shell bottom 33 toward the downstream expansion chamber 36. It subsequently enters inside the ventilation chamber 50. Between the admission inside the chamber and the ventilation chamber 50, the air is drawn in by the negative pressure generated by the fan. Upstream of the fan, the device thus defines an upstream air circulation zone, extending between the inlet 10i and the ventilation chamber 50, respectively, through the inlet tube 23 and the various parts of the shell 30. The air is subsequently blown by the fan. It exits the ventilation chamber through the opening 435 of the third membrane 43 and flows through the outlet tube 24 to the lower section 13 of the enclosure 10. Downstream of the fan, the device thus defines a downstream air circulation zone, extending between the ventilation chamber 50 and the outlet 10o, in particular, through the outlet tube 24.

In the lower section 13 of the enclosure 10, as previously stated, the air can be humidified in a humidifier, before exiting the enclosure 10 via the outlet opening 10o. It is then directed toward the respiratory mask 3, through the flexible pipe (conduit 2).

The round shape of the main components of the device 1 shown in FIGS. 4A and 4B is to be noted. It particularly involves the upper cover 21 and the shell 30. Such a shape promotes a variation of the section through which the air flows. This particularly allows successive wide portions and narrow portions to be provided along the entire length of the air circulation path through the enclosure 10. As previously indicated, this reduces the noise of the air flow. This noise can be attenuated further by arranging foam along the walls of the upper cover 21 or the internal wall of the shell 30. The round shape also increases the stiffness.

It can be seen that the device comprises a reduced number of essential components and that they are simply shaped. This allows production using 3D printing techniques or molding at a controlled cost. In the example shown, the inlet tube 23 and the outlet tube 24 are produced on the same part as the intermediate part 32 of the shell. Moreover, the first and second membranes fulfil multiple functions, as previously described. The shape of these membranes is relatively simple, which facilitates the manufacturing thereof. The device thus can be produced at a lower manufacturing cost.

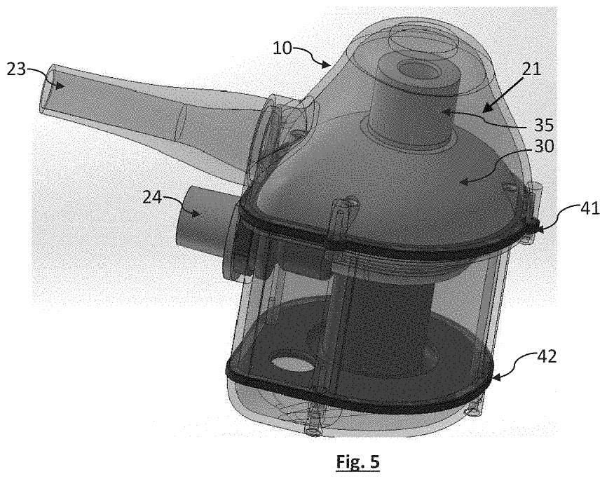

FIG. 5 schematically shows a second embodiment, similar to the embodiment described with reference to FIGS. 1 to 4. A notable difference is the orientation of the inlet tube 23 and of the outlet tube 24, with the tubes extending perpendicular to the longitudinal axis Z.

1-16. (canceled)

17. A respiratory ventilation device, configured to send an air flow, generated by a fan, into a conduit, with the conduit extending between the respiratory ventilation device and a respiratory mask configured to be used by a user, the respiratory ventilation device comprising an enclosure extending around a longitudinal axis, the enclosure comprising:
an air inlet configured to admit air into the enclosure;
an upstream air circulation zone extending between the air inlet and the fan;
an air outlet, the air outlet being configured to be connected to the conduit, so that, when the fan operates, air successively flows from the air inlet through the upstream air circulation zone, the fan and the air outlet, wherein:
the fan is arranged in a ventilation chamber;
the ventilation chamber is retained by a first flexible membrane extending between a first periphery and the ventilation chamber, the first periphery being fixedly retained relative to the enclosure, the first membrane defining a first central opening through which the ventilation chamber, or a support connected to the ventilation chamber, extends; and
wherein the ventilation chamber is retained by a second flexible membrane, separate from the first membrane, the second membrane extending between a second periphery and the ventilation chamber, the second periphery being fixedly retained relative to the enclosure at a non-zero distance from the first periphery, the second membrane comprising a second central opening, through which the ventilation chamber, or a support connected to the ventilation chamber, extends.

18. The respiratory ventilation device of claim 17, wherein:
the ventilation chamber comprises an intake portion, a fan housing, a discharge portion and a motor compartment;
the first membrane extends between the first periphery and the fan housing or between the first periphery and the motor compartment; and
the second membrane extends between the second periphery and the intake portion.

19. The respiratory ventilation device of claim 18, further comprising a third membrane extending between a third periphery, which is fixed relative to the enclosure, and the discharge portion.

20. The respiratory ventilation device of claim 17, further comprising a shell, extending around a central axis, the shell being arranged in the enclosure by being fixed relative thereto, the shell extending around the ventilation chamber, the shell defining an intake opening allowing air to be admitted into the shell, and an extraction opening allowing air to be extracted from the shell, so that the upstream air circulation zone extends:
outside the shell, between the air inlet of the enclosure and the intake opening; and
inside the shell, between the intake opening and the ventilation chamber.

21. The respiratory ventilation device of claim 20, wherein:
the shell comprises a shell bottom opposite the intake opening; and
the shell bottom faces the intake portion of the ventilation chamber with the shell bottom forming an air deflector between the intake opening and the intake portion.

22. The respiratory ventilation device of claim 21, wherein:
the central axis of the shell corresponds to the longitudinal axis; and
the intake opening and the shell bottom are aligned along the longitudinal axis.

23. The respiratory ventilation device of claim 20, wherein:
the first membrane is retained by the shell and extends between the shell and the ventilation chamber; and
the first membrane at least partly extends perpendicular to the central axis of the shell.

24. The respiratory ventilation device of claim 23, wherein:
the first membrane forms a first partition inside the shell; and
the first membrane defines an opening so as to allow air to pass on either side of the first partition.

25. The respiratory ventilation device of claim 20, wherein:
the second membrane is retained by the shell and extends between the shell and the ventilation chamber; and
the second membrane at least partly extends perpendicular to the central axis of the shell.

26. The respiratory ventilation device of claim 25, wherein:
the second membrane forms a second partition inside the shell; and
the second membrane defines an opening so as to allow air to move on either side of the second partition.

27. The respiratory ventilation device of claim 20, wherein the discharge portion of the ventilation chamber:
emerges in the extraction opening of the shell; or
extends through the extraction opening of the shell.

28. The respiratory ventilation device of claim 20, further comprising an upstream expansion chamber, adjacent to the intake opening of the shell, wherein:
the upstream expansion chamber comprises an inlet, a central part and an outlet arranged around an upstream axis; and
the section of the central part of the upstream expansion chamber, perpendicular to the upstream axis, is greater than the respective sections of the inlet and of the outlet of the upstream expansion chamber.

29. The respiratory ventilation device of claim 28, wherein the upstream expansion chamber emerges at the intake opening of the shell.

30. The respiratory ventilation device of claim 18, further comprising a downstream expansion chamber, adjacent to the intake portion of the ventilation chamber, wherein:
the downstream expansion chamber comprises an inlet, a central part and an outlet arranged around a downstream axis; and
the section of the central part of the downstream expansion chamber, perpendicular to the downstream axis, is greater than the respective sections of the inlet and gf the outlet of the downstream expansion chamber.

31. The respiratory ventilation device of claim 30, wherein:
the downstream expansion chamber emerges at the intake portion of the ventilation chamber; and
the second membrane extends between the second periphery and the downstream expansion chamber, so that the intake portion is retained by the second membrane.

32. The respiratory ventilation device of claim 17, wherein the first membrane and the second membrane are flexible and are formed from an elastomer material.
